(19) United States
(12) Patent Application Publication
(10) Pub. No.: US 2013/0171092 A1
(43) Pub. Date: Jul. 4, 2013

(54) ALPHA-CYANOACRYLATE ESTER SYNTHESIS

(71) Applicant: Henkel Ireland Limited, Monheim (DE)

(72) Inventor: Stephen Hynes, Dublin (IE)

(73) Assignee: Henkel Ireland Limited, Monheim (DE)

(21) Appl. No.: 13/777,214

(22) Filed: Feb. 26, 2013

Related U.S. Application Data
(63) Continuation of application No. PCT/EP2010/064692, filed on Oct. 1, 2010.

(51) Int. Cl. C07C 253/30; A61K 31/74

(52) U.S. Cl. 424/78.27; 558/441

(57) ABSTRACT

The high temperatures required for cracking the cyanoacrylate oligomers, produced by the Knovenagel condensation of formaldehyde and a cyanoacetate, limit the synthetic diversity and the number of different side chains that can be incorporated into a cyanoacrylate prepared using this method. Accordingly, the diversity of cyanoacrylate monomers prepared industrially is quite limited. Disclosed herein is a method for the preparation of alpha-Cyanoacrylate ester monomers from a variety of phosphonium and ammonium alpha-cyanoacrylate salts. The phosphonium and ammonium alpha-cyanoacrylate salts are of the general formula:

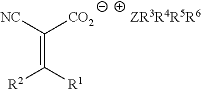

### FIELD OF THE INVENTION

The present invention relates to a novel synthetic method for providing alpha-cyanoacrylate ester monomers.

### BACKGROUND TO THE INVENTION

Traditionally, the industrial synthesis of cyanoacrylate monomers is based around the Knovenagel condensation of formaldehyde and a cyanoacetate. The presence of a basic nucleophile in the reaction mixture results in in-situ polymerisation of any cyanoacrylate monomer upon formed. In order to isolate the cyanoacrylate monomer the polymer needs to be cracked and forms a crude mixture of the monomer and bits of the broken-up polymer. The monomer is purified by distilling it off from the crude mixture. The rest of the mixture is recycled and cracked again until all of the pure monomer is retrieved.

The high temperatures required for cracking the oligomers limit the synthetic diversity and the number of different side chains that can be incorporated into a cyanoacrylate prepared using this method. Accordingly, the diversity of cyanoacrylate monomers prepared industrially is quite limited.

For example, German Patent No. DE3415181 discloses the preparation of alpha-cyanoacrylate derivates by means of thermolysis-pyrolysis at temperatures between 350-800° C. and in particular 500-750° C.

Alternative methods for the synthesis of cyanoacrylates also exist. U.S. Pat. No. 5,703,267 communicates a synthetic method for the production of alpha-cyanoacrylate esters by means of transesterification of an existing alpha-cyanoacrylate monomer. The substrate diversity of this method is limited on account of the harsh transesterification step.

International Patent Application Publication No. WO94/15907 discloses a process for the preparation of 2-cyanoacrylate esters comprising reacting 2-cyanoacrylic acid or an acid halide thereof with an alcohol in the presence of an acid catalyst. The reaction is carried out in an inert organic solvent under polymerisation inhibiting conditions and any water or hydrophilic acid generated during the reaction is continually removed. This method suffers in that the preparation of an acid halide is an additional undesirable synthetic step.

Notwithstanding the state of the art there remains a need for alternative methods for the production of alpha-cyanoacrylate ester monomers allowing increased diversity in the ester side chain.

### SUMMARY OF THE INVENTION

The present invention provides methods for the production of alpha-cyanoacrylate ester monomers. Adhesive compositions containing alpha-cyanoacrylate ester monomers are normally very rapid setting as they generally harden after a few seconds and exhibit moderate initial bond strengths. The quick setting nature of cyanoacrylate adhesives is utilised to quickly bond a variety of materials including plastics, metals, and ceramics amongst others. Accordingly, cyanoacrylate adhesives are widely used on both a domestic level and industrially, for example in the automotive, medical, and electronics industries.

The present invention provides for alpha-cyanoacrylate ester monomers that may have highly diverse functional groups on account of the relatively moderate reaction conditions required utilised in the present invention. It is envisaged that such functionalised alpha-cyanoacrylate ester monomers may provide for improved adhesive performance.

Accordingly, in a first aspect the present invention provides for use of an alpha-cyanoacrylate salt of the formula:

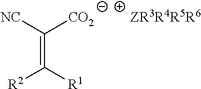

in the synthesis of alpha-cyanoacrylate ester monomers, wherein:

Z is N or P;

R¹ and R² are the same or different and are selected from H, C₁-C₂₀ aliphatic, C₃-C₂₀ cycloaliphatic, C₅-C₂₀ aromatic, C₃-C₂₀ heteroaromatic and combinations thereof;

R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or

any two of R³, R⁴, R⁵ and R⁶ may together with Z define a C₅-C₂₀ aliphatic heterocycle; or

any three of R³, R⁴, R⁵ and R⁶ may together with Z define a C₅-C₂₀ aliphatic heterocycle.

The alpha-cyanoacrylate salts of the formula given above may be synthesised utilising a method similar to that described by Krawczyk in Krawczyk, H Synth. Commun. 2000, 30, 4, 657-664. The identity of the cationic species may be readily changed by subjecting the salt to any standard cation exchange process known by a person skilled in the art.

As used herein, the term Cₓ-C_{y} aliphatic refers to linear, branched, saturated and unsaturated hydrocarbon chains comprising Cₓ-C_{y} carbon atoms (and includes Cₓ-C_{y} alkyl, Cₓ-C_{y} alkenyl and Cₓ-C_{y} alkynyl). The carbon atoms of the hydrocarbon chain may optionally be substituted one or more times with at least one of a cyano group, a nitro group, a halogen, a C₁-C₁₀ ether, a C₁-C₁₀ thioether, a C₁-C₁₀ ester, C₁-C₁₀ ketone, C₁-C₁₀ ketimine, C₁-C₁₀ sulfone, C₁-C₁₀ sulfoxide, a C₁-C₁₀ primary amide or a C₁-C₂₀ secondary amide.

Similarly, references to Cₓ-C_{y} alkyl, Cₓ-C_{y} alkenyl and Cₓ-C_{y} alkynyl include linear and branched Cₓ-C_{y} alkyl, Cₓ-C_{y} alkenyl and Cₓ-C_{y} alkynyl optionally substituted one or more times with at least one of a cyano group, a nitro group, a halogen, a C₁-C₁₀ ether, a C₁-C₁₀ thioether, a C₁-C₁₀ ester, C₁-C₁₀ ketone, C₁-C₁₀ ketimine, C₁-C₁₀ sulfone, C₁-C₁₀ sulfoxide, a C₁-C₁₀ primary amide or a C₁-C₂₀ secondary amide.

As used herein, the term “Cₓ-C_{y} cycloaliphatic” refers to unfused, fused, spirocyclic, polycyclic, saturated and unsaturated hydrocarbon rings comprising C_{X}-C_{y} carbon atoms (and includes Cₓ-C_{y} cycloalkyl, Cₓ-C_{y} cycloalkenyl and Cₓ-C_{y} cycloalkynyl). The carbon atoms of the hydrocarbon ring may optionally be replaced with at least one of O or S at least one or more times. The carbon atoms of the hydrocarbon ring may optionally be substituted one or more times with at least one of a cyano group, a nitro group, a halogen, a C₁-C₁₀ ether, a C₁-C₁₀ thioether, a C₁-C₁₀ ester, C₁-C₁₀ ketone, C₁-C₁₀ ketimine, C₁-C₁₀ sulfone, C₁-C₁₀ sulfoxide, a C₁-C₁₀ primary amide or a C₁-C₂₀ secondary amide.

Similarly, references to Cₓ-C_{y} cycloalkyl, Cₓ-C_{y} cycloalkenyl and Cₓ-C_{y} cycloalkynyl embrace compounds in which the carbon atoms of the cycloalkyl, cycloalkenyl and cycloalkynyl ring may optionally be replaced with at least one of O or S at least one or more times. The carbon atoms of the rings may optionally substituted one or more times with at least one of a cyano group, a nitro group, a halogen, a C₁-C₁₀ ether, a C₁-C₁₀ thioether, a C₁-C₁₀ ester, C₁-C₁₀ ketone, C₁-C₁₀ ketimine, C₁-C₁₀ sulfone, C₁-C₁₀ sulfoxide, a C₁-C₁₀ primary amide or a C₁-C₂₀ secondary amide.

As used herein, the term aromatic refers to a aromatic carbocyclic structure in which the carbon atoms of the aromatic ring may optionally be substituted one or more times with at least one of a cyano group, a nitro group, a halogen, a C₁-C₁₀ ether, a C₁-C₁₀ thioether, a C₁-C₁₀ ester, C₁-C₁₀ ketone, C₁-C₁₀ ketimine, C₁-C₁₀ sulfone, C₁-C₁₀ sulfoxide, a C₁-C₁₀ primary amide or a C₁-C₂₀ secondary amide.

As used herein, the term heterocycle refers to cyclic compounds having as ring members atoms of at least two different elements.

As used herein, the term heteroaromatic refers to an aromatic heterocyclic structure having as ring members atoms of at least two different elements. The carbon atoms of the heteroaromatic ring may optionally be substituted one or more times with at least one of a cyano group, a nitro group, a halogen, a C₁-C₁₀ ether, a C₁-C₁₀ thioether, a C₁-C₁₀ ester, C₁-C₁₀ ketone, C₁-C₁₀ ketimine, C₁-C₁₀ sulfone, C₁-C₁₀ sulfoxide, a C₁-C₁₀ primary amide or a C₁-C₂₀ secondary amide

The variables R¹ and R² may be H. For example, Z may be N and R¹ and R² may be H.

The variable R³ may be H. Both R³ and R⁴ may be H. When both R³ and

R⁴ are H, R⁵ and R⁶ may be the same or different and may be selected from C₃-C₂₀ alkyl and C₃-C₂₀ cycloalkyl. Cationic phosphonium or ammonium species possessing steric bulk may provide for more stable alpha-cyanoacrylate salts. In one embodiment Z may be N, R¹ and R² may be H, R³ and R⁴ may H, and R⁵ and R⁶ may be the same or different and may be selected from C₃-C₂₀ alkyl and C₃-C₂₀ cycloalkyl.

Alternatively, when both R³ and R⁴ are H, R⁵ and R⁶ may be the same or different and may be selected from a cyclohexyl moiety, an iso-propyl moiety, an iso-butyl moiety, and a tert-butyl moiety. R³ and R⁴ may be H, and R⁵ and R⁶ may be a cyclohexyl moiety. In one embodiment Z may be N, R¹ and R² may be H, R³ and R⁴ may H, and R⁵ and R⁶ may be the same or different and may be selected from a cyclohexyl moiety, an iso-propyl moiety, an iso-butyl moiety, and a tert-butyl moiety.

The alpha-cyanoacrylate salt may be of the formula:

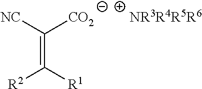

wherein:

R¹ and R² are the same or different and are selected from H, C₁-C₂₀ aliphatic, C₃-C₂₀ cycloaliphatic, C₅-C₂₀ aromatic, C₃-C₂₀ heteroaromatic and combinations thereof;

R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or

any two of R³, R⁴, R⁵ and R⁶ may together with N define a C₅-C₂₀ aliphatic heterocycle; or

any three of R³, R⁴, R⁵ and R⁶ may together with N define a C₅-C₂₀ aliphatic heterocycle.

Advantageously, cationic ammonium counterions may provide for more stable alpha-cyanoacrylate salts.

The variables R¹ and R² may be H. The variable R³ may be H. Both R³ and R⁴ may be H. When both R³ and R⁴ are H, R⁵ and R⁶ may be the same or different and may be selected from C₃-C₂₀ alkyl and C₃-C₂₀ cycloalkyl.

In one embodiment R¹ and R² may be H, R³ and R⁴ may H, and R⁵ and R⁶ may be the same or different and may be selected from C₃-C₂₀ alkyl and C₃-C₂₀ cycloalkyl.

Alternatively, when both R³ and R⁴ are H, R⁵ and R⁶ may be the same or different and may be selected from a cyclohexyl moiety, an iso-propyl moiety, an iso-butyl moiety, and a tert-butyl moiety. R³ and R⁴ may be H, and R⁵ and R⁶ may be a cyclohexyl moiety.

The alpha-cyanoacrylate salt may be of the formula:

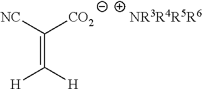

wherein:

R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or

any two of R³, R⁴, R⁵ and R⁶ may together with N define a C₅-C₂₀ aliphatic heterocycle; or

any three of R³, R⁴, R⁵ and R⁶ may together with N define a C₅-C₂₀ aliphatic heterocycle.

The variable R³ may be H. Both R³ and R⁴ may be H. When both R³ and R⁴ are H, R⁵ and R⁶ may be the same or different and may be selected from C₃-C₂₀ alkyl and C₃-C₂₀ cycloalkyl. Alternatively, when both R³ and R⁴ are H, R⁵ and R⁶ may be the same or different and may be selected from a cyclohexyl moiety, an iso-propyl moiety, an iso-butyl moiety, and a tert-butyl moiety. R³ and R⁴ may be H, and R⁵ and R⁶ may be a cyclohexyl moiety.

The alpha-cyanoacrylate salt may be of the formula:

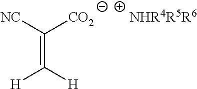

wherein:

R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R⁴, R⁵ and R⁶ are not H; or

any two of R⁴, R⁵ and R⁶ may together with N define a C₅-C₂₀ aliphatic heterocycle; or R⁴, R⁵ and R⁶ may together with N define a C₅-C₂₀ aliphatic heterocycle.

The variable R⁴ may be H. When R⁴ is H, R⁵ and R⁶ may be the same or different and may be selected from C₃-C₂₀ alkyl and C₃-C₂₀ cycloalkyl. Alternatively, when R⁴ is H, R⁵ and R⁶ may be the same or different and may be selected from a cyclohexyl moiety, an iso-propyl moiety, an iso-butyl moiety, and a tert-butyl moiety. R⁴ may be H, and R⁵ and R⁶ may be a cyclohexyl moiety.

In a further aspect, the present invention provides for a method of preparing an alpha-cyanoacrylate ester monomer comprising the step of:

reacting an alpha-cyanoacrylate salt of the general formula:

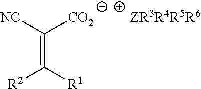

with a compound of the general formula R⁷—X, wherein:

Z is N or P;

R¹ and R² are the same or different and are selected from H, C₁-C₂₀ aliphatic, C₃-C₂₀ cycloaliphatic, C₅-C₂₀ aromatic, C₃-C₂₀ heteroaromatic and combinations thereof;

R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or

any two of R³, R⁴, R⁵ and R⁶ may together with Z define a C₅-C₂₀ aliphatic heterocycle; or

any three of R³, R⁴, R⁵ and R⁶ may together with Z define a C₅-C₂₀ aliphatic heterocycle;

R⁷ is selected from the group consisting of C₁-C₂₀ aliphatic, C₃-C₂₀ cycloaliphatic and combinations thereof; and

X is a leaving group, wherein the conjugate acid HX of the leaving group X has a pKₐ of −2 or less.

References to pKₐ within this specification are to be construed as pKₐ (H₂O). In particular to pKₐ measurements carried out at 25±1° C. in distilled water solutions (i.e., non ionic-strength-adjusted distilled water solutions). The pKₐ value indicated refers to the pKₐ of the first removable proton of the acid.

As used herein, the term “leaving group” refers to species that departs with a pair of electrons in heterolytic bond cleavage.

The variable Z may be N. The variables R¹ and R² may be H. Z may be N and R¹ and R² may be H.

The variable R³ may be H. Both R³ and R⁴ may be H. When both R³ and R⁴ are H, R⁵ and R⁶ may be the same or different and may be selected from C₃-C₂₀ alkyl and C₃-C₂₀ cycloalkyl. In one embodiment Z may be N, R¹ and R² may be H, R³ and R⁴ may H, and R⁵ and R⁶ may be the same or different and may be selected from C₃-C₂₀ alkyl and C₃-C₂₀ cycloalkyl.

Alternatively, when both R³ and R⁴ are H, R⁵ and R⁶ may be the same or different and may be selected from a cyclohexyl moiety, an iso-propyl moiety, an iso-butyl moiety, and a tert-butyl moiety. R³ and R⁴ may be H, and R⁵ and R⁶ may be a cyclohexyl moiety. In one embodiment Z may be N, R¹ and R² may be H, R³ and R⁴ may H, and R⁵ and R⁶ may be the same or different and may be selected from a cyclohexyl moiety, an iso-propyl moiety, an iso-butyl moiety, and a tert-butyl moiety.

The alpha-cyanoacrylate salt may be of the formula:

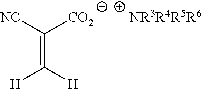

wherein:

R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or

any two of R³, R⁴, R⁵ and R⁶ may together with N define a C₅-C₂₀ aliphatic heterocycle; or

any three of R³, R⁴, R⁵ and R⁶ may together with N define a C₅-C₂₀ aliphatic heterocycle.

The variable R³ may be H. Both R³ and R⁴ may be H. When both R³ and R⁴ are H, R⁵ and R⁶ may be the same or different and may be selected from C₃-C₂₀ alkyl and C₃-C₂₀ cycloalkyl. Alternatively, when both R³ and R⁴ are H, R⁵ and R⁶ may be the same or different and may be selected from a cyclohexyl moiety, an iso-propyl moiety, an iso-butyl moiety, and a tert-butyl moiety. R³ and R⁴ may be H, and R⁵ and R⁶ may be a cyclohexyl moiety.

The alpha-cyanoacrylate salt may be of the formula:

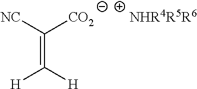

wherein:

R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R⁴, R⁵ and R⁶ are not H; or

any two of R⁴, R⁵ and R⁶ may together with N define a C₅-C₂₀ aliphatic heterocycle; or R⁴, R⁵ and R⁶ may together with N define a C₅-C₂₀ aliphatic heterocycle.

The variable R⁴ may be H. When R⁴ is H, R⁵ and R⁶ may be the same or different and may be selected from C₃-C₂₀ alkyl and C₃-C₂₀ cycloalkyl. Alternatively, when R⁴ is H, R⁵ and R⁶ may be the same or different and may be selected from a cyclohexyl moiety, an iso-propyl moiety, an iso-butyl moiety, and a tert-butyl moiety. R⁴ may be H, and R⁵ and R⁶ may be a cyclohexyl moiety.

With reference to the compound of the formula R⁷—X, R⁷ may be C₁-C₂₀ aliphatic. For example, R⁷ may be C₁-C₂₀ alkyl.

The variable X may be selected from the group Cl, Br, I, (p)-CH₃C₆H₄SO₃, CH₃SO₃, ClO₄, CF₃SO₃ and FSO₃. For example, X may be selected from the group (p)-CH₃C₆H₄SO₃, CH₃SO₃, ClO₄, CF₃SO₃ and FSO₃.

Desirably, the conjugate acid HX of the leaving group X may have a pKₐ of between −8 and −20. For example a pKₐ of between −10 and −18. In particular, the conjugate acid HX of the leaving group X may have a pKₐ of between −12 and −18. Suitably, the conjugate acid HX of the leaving group X may have a pKₐ of between −12 and −16. Suitably, X may be selected from the group consisting of CF₃SO₃ and FSO₃. X may be CF₃SO₃.

Advantageously, by choosing a leaving group X with a suitable pKₐ the method of the present invention allows for the efficient synthesis of alpha-cyanoacrylate ester monomers. The method of the present invention proceeds with high chemoselectivity and minimal by-products are observed. Furthermore, unwanted polymerisation of the alpha-cyanoacrylate ester monomers produced by the method of the present invention is minimised by appropriate selection of the leaving group X (and its associated pKₐ).

The step of reacting the alpha-cyanoacrylate salt with a compound of the general formula R⁷—X according to the method of the present invention may be carried out in a solvent selected from the group consisting of C₂-C₂₀ acyclic ethers, C₅-C₂₀ cyclic ethers, C₁-C₂₀ haloalkyl, C₂-C₂₀ alkylnitriles, C₃-C₂₀ alkylesters, C₅-C₂₀ alkanes and combinations thereof.

Desirably, the solvent is C₁-C₂₀ haloalkyl. For example, the solvent may be C₁-C₁₀ chloroalkyl. Suitable solvents include dichloromethane.

Moreover, the step of reacting the alpha-cyanoacrylate salt with a compound of the general formula R⁷—X according to the method of the present invention may be carried out at a temperature between −20° C. and 60° C.+60° C.). For example, the step of reacting the alpha-cyanoacrylate salt with a compound of the general formula R⁷—X according to the method of the present invention may be carried out at a temperature between 15° C. and 25° C. In particular, a temperature of 22° C. may be desirable.

The alpha-cyanoacrylate monomers prepared according to the present invention may be isolated or purified by any conventional technique known by the person skilled in the art. For example, purification may be carried out by distillation, chromatography or crystallisation where appropriate.

Where alpha-cyanoacrylate monomers prepared according to the present invention are intended for use in medical or surgical applications the monomer may be sterilised, for example by means of irradiation, prior to use. Sterilisation may be effected in the presence of a stabilizer so as to prevent polymerisation during the sterilisation process.

Alpha-cyanoacrylate monomers prepared according to the present invention may be formulated as part of an adhesive composition together with additives selected from the group consisting of plasticizers, accelerators, fillers, opacifiers, thickeners, viscosity modifiers, inhibitors, thixotrophy conferring agents, stabilizers, dyes, and combinations thereof.

In particular, such cyanoacrylate compositions may contain thickeners as further auxiliary substances. This is desirable especially when the composition is utilised to bond porous materials which otherwise readily absorb the low viscosity adhesive. Suitable thickners may include polymethyl methacrylate, methacrylate copolymers, acrylic rubber, cellulose derivatives, polyvinyl acetate or polyalphacyanoacrylate.

Normally, stabilizer systems have to be selected so that no polymerisation occurs during transportation and storage of the cyanoacrylate composition. Whereas, after application of the composition to a desired substrate polymerisation will occur immediately. Accordingly, besides known radical polymerisation inhibitors, inhibitors against anionic polymerisation are generally added to cyanoacrylate adhesives.

Where suitable, it will be appreciated that all optional and/or preferred features of one embodiment of the invention may be combined with optional and/or preferred features of another/other embodiment(s) of the invention.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

It should be readily apparent to one of ordinary skill in the art that the examples disclosed herein below represent generalised examples only, and that other arrangements and methods capable of reproducing the invention are possible and are embraced by the present invention.

### EXAMPLE 1

### General Synthetic Procedure: Synthesis of 1-Octyl Alpha-Cyanoacrylate by Alkylation of Dicyclohexylammonium Alpha-Cyanoacrylate

The synthesis of dicyclohexylammonium alpha-cyanoacrylate can be found in the following reference: Krawczyk, H Synth. Commun. 2000, 30, 4, 657-664.

To a solution of octyl triflate (0.05 mol) in dry dichloromethane (DCM, 100 mL) was added trifluoroacetic acid [TFA] (0.004 mol) and t-butylated hydroxyanisole (0.25 wt %). A solution of Dicyclohexylammonium alpha-cyanoacrylate in dry DCM (80 mL) was added dropwise (2 h). The resulting solution was stirred at 22° C. overnight. NMR showed approx 10% of the triflate remaining with no trace of polymer. A solution of 0.078 mol of Dicyclohexylammonium alpha-cyanoacrylate in dry DCM (20 mL) was added. The mixture was stirred at 22° C. until triflate had disappeared in the NMR (4 h). The mixture was acidified (TFA) and reduced in vacuo. The precipitated solid was removed by filtration. Hexane (100 mL) was added. The mixture was reduced and filtered again. This was repeated. The solvents were removed to afford 11.5 g of a yellow liquid which was −75-80% CA monomer. The impurities are cyanoacrylic acid (due to the excess of Dicyclohexylammonium alpha-cyanoacrylate used), dioctyl ether (a byproduct carried through from the triflate formation) a small amount (<2%) of polymer, trace amounts of the amine triflate salt, octyl cyanoacetate (formed from cyanoacetic acid amine salt, a residue from incomplete formation of the Dicyclohexylammonium alpha-cyanoacrylate). There are no byproducts or side reaction evident from the CA formation reaction. The octyl CA was distilled to purify and afforded an overall yield of 65% based on triflate. The synthesis procedure is general and has been used to prepare crude samples of other monomers such as n-propyl CA, 3-methoxybutyl CA and bis-cyanoacrylic acid ester of PEG 400.

NMR Analysis of distilled material: ¹H NMR (CDCl₃) δ: 7.05, (s, 1H, ═CHH); 6.65 (s, 1H, ═CHH); 4.27 (t, 2H, ˜COOCH₂CH₂˜); 1.73 (m, 2H, ˜COOCH₂CH₂˜); 1.40-1.25 (br m, 10H, ˜COOCH₂CH₂CH₂CH₂ CH₂CH₂CH₂˜); 0.88 (t, 3H, ˜CH₂CH₃). ¹³C δ: 163.27 ˜COO˜; 143.3, ═CH₂; 114.5, ˜CN; 113.4, ˜C═CH₂; 66.99, ˜COOCH₂˜; 31.8; 29.2; 28.4; 25.8; 24.7; 22.7 (˜CH₂CH₃); 14.11 (˜CH₃).

The general reaction scheme for the reaction of reaction of Dicyclohexylammonium alpha-cyanoacrylate with electrophiles is given below. All alkyl triflates were prepared immediately prior to use and used crude. Unless otherwise indicated in the Reaction Conditions Column in Tables 1-4, the general synthetic procedure indicated above was followed for each reaction.

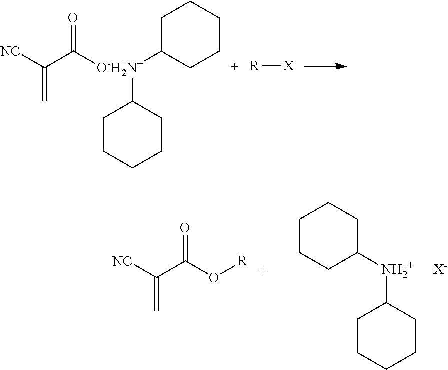

TABLE 1

| R (or alcohol | | | | |
|---|---|---|---|---|
| from which R | | Reaction | | Monomer |
| is derived) | X | Conditions | Result | Yield |
| n-propanol | OTf | DCM, overnight, | Monomer apparent by | ~40% |
| | | 22° C. | NMR and intact upon | |
| | | | isolation but only when | |
| | | | reaction mixture | |
| | | | stabilised with TFA. | |
| | | | Polymerisation occurred | |
| | | | in the absence of TFA | |
| n-propanol | OTf | CDCl₃/d⁶-acetone, | Reaction monitored in | Crude |
| | | 30 min, 22° C. | deuterated solvent | monomer |
| | | | | isolated in |
| | | | | 80% yield |
| n-octanol | OTf | DCM, 22° C., | | Crude yield |
| | | heptane and TFA | | 74% |
| | | (0.25 equiv) | | |
| | | added after | | |
| | | reaction | | |
| | | completion | | |
| n-octanol | OTf | DCM, 22° C., | | Crude yield |
| | | heptane and TFA | | 74% |
| | | (0.25 equiv) | | |
| | | added after | | |
| | | reaction | | |
| | | completion | | |

TABLE 2

| R (or alcohol | | | | |
|---|---|---|---|---|
| from which R | | | | Monomer |
| is derived) | X | Reaction Conditions | Result | Yield |
| n-octanol | OTf | DCM, 22° C. overnight, | | Crude yield |
| | | with 1.05 equiv of | | 100% based |
| | | CA salt. 30 mol % | | on triflate |
| | | TFA added after | | purity of 90% |
| | | reaction completion | | |
| n-octanol (a) | OTf | DCM, 22° C. overnight, | Crude stabilised | 72% after |
| | | 30% TFA added after | with MSA prior to | distillation |
| | | reaction completion | distillation | |
| PEG400 (a) | OTf | 2 days at 22° C. in | Monomer visible | N/A |
| | (bis) | DCM, then TFA, | by NMR | |
| | | heptane | | |
| 3-methoxy | OTf | DCM, 22° C. 20 h, | Crude monomer | 66% |
| butanol (a) | | TFA (30 mol %) added | (70% pure) was | |
| | | upon reaction | flash | |
| | | completion | chromatographed | |
| | | | to afford 66% of | |
| | | | pure product | |
| | | | (98% by GC) | |
| 3-methoxy | OTf | DCM, 22° C., 24 h, | Crude yield 68%, | Pure yield |
| butanol | | TFA 10 mol % added | 80% purity | 40% after |
| | | before workup | Contamination | flash |
| | | | with | chromatography |
| | | | cyanoacetate | to afford |
| | | | | 95% pure |
| | | | | product |

(a) Purification can be carried out via distillation, chromatography or crystallisation where appropriate.

TABLE 3

| R (or alcohol | | | | |
|---|---|---|---|---|
| from which R | | | | Monomer |
| is derived) | X | Reaction Conditions | Result | Yield |
| 1-octanol | OTf | DCM, 22° C., overnight, | Crude monomer 83% | Flash |
| | | TFA added (30 mol %) | yield 89% purity | chromatography |
| | | with workup | | afforded 42% |
| | | | | yield of >96% |
| | | | | purity |
| PEG400 | OTf | 2 days at 22° C. in | Monomer visible | N/A |
| | (bis) | DCM, then TFA, | by NMR | |
| | | heptane | | |
| 3-methoxy | OTf | DCM, 22° C. 20 h, | Crude monomer (70% | 66% |
| butanol | | TFA (30 mol %) added | pure) was flash | |
| | | upon reaction | chromatographed to | |
| | | completion | afford 66% of pure | |
| | | | product (98% by GC) | |
| polidocanol | OTf | CD₃CN, overnight, | Monomer visible | |
| | | 22° C. | by NMR | |
| 1,4 butane | OTf | CD₃CN/CDCl₃, 50° C. 3 h | Bis CA monomer | |
| diol | | | visible by NMR | |
| polidocanol | OTf | DCM, 15 h, 22° C. | Reaction complete | |
| | | | by NMR | |

What is claimed is:

1. A method of preparing an alpha-cyanoacrylate ester monomer comprising the step of:
reacting an alpha-cyanoacrylate salt of the general formula: 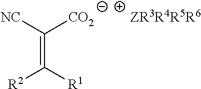
with a compound of the general formula R⁷—X, wherein:
Z is N or P;
R¹ and R² are the same or different and are selected from H, C₁-C₂₀ aliphatic, C₃-C₂₀ cycloaliphatic, C₅-C₂₀ aromatic, C₃-C₂₀ heteroaromatic and combinations thereof;
R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or
any two of R³, R⁴, R⁵ and R⁶ may together with Z define a C₅-C₂₀ aliphatic heterocycle; or
any three of R³, R⁴, R⁵ and R⁶ may together with Z define a C₅-C₂₀ aliphatic heterocycle;
R⁷ is selected from the group consisting of C₁-C₂₀ aliphatic, C₃-C₂₀ cycloaliphatic and combinations thereof; and
X is a leaving group, wherein the conjugate acid HX of the leaving group X has a pKₐ of −2 or less.

2. A method according to claim 1 wherein Z is N.

3. A method according to claim 1 wherein R¹ and R² are H.

4. A method according to claim 1 wherein R³ is H.

5. A method according to claim 1 wherein R³ and R⁴ are H.

6. A method according to claim 5 wherein R⁵ and R⁶ are the same or different and are selected from C₃-C₂₀ alkyl and C₃-C₂₀ cycloalkyl.

7. A method according to claim 6 wherein R⁵ and R⁶ are the same or different and are selected from the group consisting of a cyclohexyl moiety, an iso-propyl moiety, an iso-butyl moiety, and a tert-butyl moiety.

8. A method according to claim 1 wherein R⁷ is C₁-C₂₀ aliphatic.

9. A method according to claim 8 wherein R⁷ is C₁-C₂₀ alkyl.

10. A method according to claim 1 wherein X is selected from the group consisting of (p)-CH₃C₆H₄SO₃, CH₃SO₃, ClO₄, CF₃SO₃ and FSO₃.

11. A method according to claim 1 wherein the conjugate acid HX of the leaving group X has a pKₐ of −12 or less.

12. A method according to claim 11 wherein X is selected from the group consisting of CF₃SO₃ and FSO₃.

13. A method according to claim 12 wherein the step of reacting the alpha-cyanoacrylate salt with a compound of the general formula R⁷—X is carried out in a solvent selected from the group consisting of C₂-C₂₀ acyclic ethers, C₅-C₂₀ cyclic ethers, C₁-C₂₀ haloalkyl, C₂-C₂₀ alkylnitriles, C₃-C₂₀ alkylesters, C₅-C₂₀ alkanes and combinations thereof.

14. A method according to claim 13 wherein the solvent is C₁-C₁₀ chloroalkyl.

15. A method according to claim 14 wherein the solvent is dichloromethane.

16. A method according to claim 15 wherein the step of reacting the alpha-cyanoacrylate salt with a compound of the general formula R⁷—X is carried out at a temperature between −20° C. and 60° C.

17. A method according to claim 16 wherein the temperature is between 15° C. and 25° C.

18. Use of an alpha-cyanoacrylate salt of the formula: 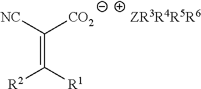
in the synthesis of alpha-cyanoacrylate ester monomers, wherein:
Z is N or P;
R¹ and R² are the same or different and are selected from H, C₁-C₂₀ aliphatic, C₃-C₂₀ cycloaliphatic, C₅-C₂₀ aromatic, C₃-C₂₀ heteroaromatic and combinations thereof;
R³, R⁴, R⁵ and R⁶ are the same or different and are selected from H, C₁-C₂₀ aliphatic, C₃-C₂₀ cycloaliphatic, C₅-C₂₀ aromatic, C₃-C₂₀ heteroaromatic and combinations thereof, such that at least two of R³, R⁴, R⁵ and R⁶ are not H; or
any two of R³, R⁴, R⁵ and R⁶ may together with Z define a C₅-C₂₀ aliphatic heterocycle; or
any three of R³, R⁴, R⁵ and R⁶ may together with Z define a C₅-C₂₀ aliphatic heterocycle.

19. Use according to claim 18 wherein Z is N.

20. Use according to claim 18 wherein R¹ and R² are H.

21. Use according to claim 18 wherein R³ is H.

22. Use according to claim 18 wherein R³ and R⁴ are H.

23. Use according to claim 22 wherein R⁵ and R⁶ are the same or different and are selected from C₃-C₂₀ alkyl and C₃-C₂₀ cycloalkyl.

24. Use according to claim 23 wherein R⁵ and R⁶ are the same or different and are selected from the group consisting of a cyclohexyl moiety, an iso-propyl moiety, an iso-butyl moiety, and a tert-butyl moiety.
